(19) United States
(12) Patent Application Publication
(10) Pub. No.: US 2011/0171281 A1
(43) Pub. Date: Jul. 14, 2011

(54) SOFT CAPSULE COMPOSITION AND METHOD OF USE

(76) Inventor: Karl Wei Cao, Richmond (CA)

(21) Appl. No.: 12/687,274

(22) Filed: Jan. 14, 2010

(51) Int. Cl. A61K 9/48; A61K 9/00; A61K 35/60; A61K 31/714; A61K 31/60; A61K 31/59; A61K 31/51; A61K 31/4415; A61K 31/455; A61K 31/355; A61K 31/167; A61K 31/07; A61K 47/36; A61K 8/02

(52) U.S. Cl. 424/422; 424/451; 424/455; 424/463; 424/523; 514/52; 514/159; 514/167; 514/276; 514/345; 514/355; 514/458; 514/629; 514/725; 514/777; 514/778; 514/779; 514/780

(57) ABSTRACT

A clear soft vegetarian dosage capsule comprising a first and second biphasic film. The first biphasic film has a plurality of first reservoirs and further comprises a polysaccharide, a seaweed extract, a rheology modifier, a buffer and a plasticizer. The second biphasic film has a plurality of second reservoirs having a formulation identical to the first film to ensure a sealing engagement. A dosage amount is disposed in each of the plurality of the reservoirs forming a plurality of filled reservoirs. The dosage amount is an emulsion, oil, hydrophilic liquid. The first film is extruded or overlaid with the second film allowing each of the plurality of second reservoirs to overlay the filled reservoirs and sealed with pressure and a fusion temperature slightly less than the melting point of the first and second films forming clear soft vegetarian dosage capsules that are blown dry, bonding moisture to the polysaccharide.

### FIELD

The present embodiments generally relate to capsules made from a vegetable based product and to methods of making a capsule using a vegetable based product.

### BACKGROUND

A need exists for a method of making soft capsules with materials other than gelatin and for soft capsules made with materials other than gelatin that are able to form hydrated solutions with the strength and elasticity required for use in rotary die processes

A further need exists for soft capsules made with materials other than gelatin that mimic the behavior and characteristics of soft capsules made with mammalian gelatin while overcoming the disadvantages of mammalian gelatin.

A need exists for a method of forming film solutions utilizing non animal ingredients during casting that are mechanically strong and exhibit elasticity sufficient to allow the film to stretch during filling to create a vegetable based soft capsule that has mechanical and dimensional stability as well as the elasticity and strength adequate for use in a continuous commercial process.

A need also exists for a method of producing a film solution to be used to from two halves of a soft capsule and that will fuse together during the filling and cutting process when subjected to sufficient pressure and elevated temperature below the melting point of the film solution.

### DETAILED DESCRIPTION OF THE EMBODIMENTS

Before explaining the present methods and composition in detail, it is to be understood that the methods and composition is not limited to the particular embodiments and that it can be practiced or carried out in various ways.

The methods and composition relate to vegetable based soft capsules that can be made using a rotary die encapsulation apparatus. The methods provide compositions that do not employ mammalian gelatin and, therefore, overcome the disadvantages associated with mammalian gelatin material.

The clear soft vegetarian dosage capsule can include a biphasic first film solution with a plurality of first reservoirs. The biphasic first film can contain at least 6 ingredients as follows: 1) a polysaccharide, 2) a seaweed extract, 3) a rheology modifier, 4) a buffer, 5) a plasticizer and 6) water.

A first phase of the biphasic network system can be created using a polysaccharide, a seaweed extract, a rheology modifier and a water, and a second phase or filler can be created for the first phase network by blending a water, a buffer, a polysaccharide, and a plasticizer at a temperature less than 80 Fahrenheit.

After the filler is formed, the filler can be mixed into the first phase network system forming a biphasic solution while maintaining a temperature of not more than 80 degrees Fahrenheit. The biphasic solution can than be processed using a rotary die process to form a first film with a plurality of reservoirs. The rotary die process can be similar to one or more rotary die process disclosed in The Theory and Practice of Industrial Pharmacy (Lachman, Lieberman and Kanig, Editors), 3.sup.rd Edition, published by Lea & Febiger), which is incorporated herein in its entirety.

A second film with a plurality of second reservoirs having a biphasic solution identical to the first film can be created in a manner similar to the first film. The second film can be joined with the first film to ensure a sealing engagement.

A dosage amount can be placed or disposed within the plurality of first reservoirs forming a plurality of filled reservoirs. The dosage amount can be an emulsion, an oil, a hydrophilic liquid, a nutraceutical, or a pharmaceutical.

The first film can be extruded or overlaid with the second film allowing each of the plurality of second reservoirs to overlay the filled reservoirs. The first film and the second film can be sealed with pressure and a fusion temperature slightly less than the melting point of the first and second films. The fusion temperature can be from about 5 degrees Fahrenheit to about 10 degrees Fahrenheit less than the melting point of the first and second films, forming a sheet of vegetarian dosage amounts which are cut into clear soft vegetarian dosage capsules upon fusion.

The cut soft vegetarian dosage capsules can be put into a chamber with a humidity that is no more than about 25 percent and the water is blown away on an outer surface of the soft capsules, the blown hot air can be at a temperature from about 70 degrees Celsius to about 77 degrees Celsius. Accordingly, water can be bonded or locked to the polysaccharide, rendering each soft capsule to have moisture level from about 10.0 percent to about 15.0 percent. This can make the outer surface of the soft capsules surprisingly resistant to humidity and moisture, while maintaining a brilliantly clear, mechanically and dimensionally stable soft capsule resistant to leaking, crosslinking, and hygroscopic material. The clear soft vegetarian and gelatin free capsule surprisingly inhibits microbial activity by this bonding or locking of the water to the polysaccharide of the clear soft vegetarian gelatin free capsule upon drying.

The clear soft vegetarian dosage capsule can contain polysaccharides. The polysaccharides can be alginates, agar gum, gellan, pullulan, starch, tara gum, locust bean gum, glucomannan, or monosaccharide gums, or combinations thereof

The clear soft vegetarian dosage capsule of the present invention can contain a seaweed extract. The seaweed extract can be carrageenan, red seaweed, brown seaweed, funoran algae, red algae, brown algae, green algae, kelp, marine plants, or combinations thereof.

The clear soft vegetarian dosage capsule of the present invention can also contain a rheology modifier. The rheology modifier can be a xanthan gum, arabic gum, karaya gum, or combinations thereof.

The clear soft vegetarian dosage capsule can further contain a buffer. The buffer can be potassium acetate, magnesium acetate, ethylenediaminetetraacetic acid, or combinations thereof.

The clear soft vegetarian dosage capsule further contains a plasticizer, which can be a glycerin, sorbitol, or glycol and combinations thereof.

The clear soft vegetarian dosage capsule conatains polysaccharide at a weight percent between 20.0 and 42.0, the seaweed exact has a weight percent from about 5.0 to about 24.0, the rheology modifier has a weight percent from about 7.0 to about 22.0, the buffer with a weight percent from about 0.01 to about 10.0, and the plasticizer has weight percent from about 10.0 to about 40.0. Each of the films further contains from about 30.0 to about 80.0 weight percent of water.

The clear soft vegetarian dosage capsule can also be used as a suppository, a wafer, a sublingual dissolvable film, a quick-dissolve ampule, a lozenge, or a strip.

The clear soft vegetarian dosage capsule can include a drug carrier. The drug carrier can be a hard capsule or a soft capsule.

The clear soft vegetarian dosage capsule can contain a liquid dosage, such as a marine oil, marine lipids, fish oil, vitamin oil, omega oil, synthetic oil, vegetable oil, and combinations thereof.

The clear soft vegetarian dosage capsule can contain a dosage amount of a pharmaceutical, such as liquid aspirin, liquid antihistamine, liquid delivery acetaminophen, liquid anti-inflammatory drugs, liquid antibiotics, and combinations thereof.

The clear soft vegetarian dosage capsule can contain a nutritional dosage amount such as Vitamin E, Vitamin B1, algae DHA, Vitamin A, Vitamin D, Vitamin B12, other B vitamin mixtures, Vitamin B-6, Vitamin B3, flax seed oil, lecithin, linoleic acids, evening primrose oil, borage oil, pumpkin seed oil, lipids, omega oils, fish oils, silymarin, or combinations thereof.

The clear soft vegetarian dosage capsule can contain a cosmetic dosage amount such as a liquid foundation, a moisturizer, an antiaging product, a skin de-wrinkle component, other minerals, or combinations thereof.

The clear soft vegetarian dosage capsule films can also contain a flavoring additive such as citrus flavoring, mint flavoring, rum flavoring, fruit flavoring, bubblegum flavoring, vanilla flavoring, cocoa flavoring, licorice flavoring, coffee flavoring, or combinations thereof.

The clear soft vegetarian dosage capsule can also contain an edible coloring in at least one of the films, such as azoquinophthalone triphenylmethane, xanthene, indigoid dyes, iron oxides, iron hydroxides, titanium dioxides, carbon black, riboflavin, carotenes, anthocyanines, tumeric, cochineal extracts, chlorophyllin, canthaxanthin, caramel, betannin, or combinations thereof.

The clear soft vegetarian dosage capsule can have a reservoir that is a size from about 100 mg to about 1500 mg.

The clear soft vegetarian dosage capsule can be made from or have a film that has a thickness when dry, and after cooling from about 0.05 cm to about 0.09 cm.

The clear soft vegetarian dosage capsule wherein the plurality of clear soft vegetarian dosage capsules are formed simultaneously with cutting, and sealing using a pressure from about 0.25 psi to about 1 psi.

The clear soft vegetarian dosage capsule can have an elasticity sufficient to allow each film to stretch during filling for use during continuous commercial processes for each film.

Each clear soft vegetarian dosage capsule contains a reservoir that has a shape of round, elliptical, or oblong.

The clear soft vegetarian dosage capsule can also be coated with a polysaccharide coating disposed over the clear soft vegetarian dosage capsules. The polysaccharide coating can be a different polysaccharide.

The term marine oils and fish extracts can include fish oil, salmon oil, cod oil, seal oil, krill oil, anchovy oil, sardine, oil mackerel oil and similar oils from marine animals, marine mammals, marine plants, and combinations thereof.

While these embodiments have been described with emphasis on the embodiments, it should be understood that within the scope of the appended claims, the embodiments might be practiced other than as specifically described herein.

What is claimed is:

1. A clear soft vegetarian dosage capsule comprising:
a. a first biphasic film with a plurality of first reservoirs, wherein the first biphasic film comprises:
i. a polysaccharide;
ii. a seaweed exact;
iii. a rheology modifier;
iv. a buffer; and
v. a plasticizer;
b. a second biphasic film with a plurality of second reservoirs having a formulation identical to the first biphasic film; is disposed at least partially about the first biphasic film; wherein the first biphasic film is extruded or overlaid with the second biphasic film and be sealed with a pressure and a fusion temperature slightly less than a melting point of the first biphasic film and the second biphasic film forming the clear soft vegetarian dosage capsules that are blown dry; and
c. wherein a dosage amount is disposed in one or more of the reservoirs of the plurality of the first reservoirs forming a plurality of filled reservoirs and wherein the dosage amount is an emulsion, oil, or hydrophilic liquid; and wherein the plurality of second reservoirs overlay the filled reservoirs.

2. The clear soft vegetarian dosage capsule of claim 1, wherein the fusion temperature is between 5 degrees Fahrenheit to 10 degrees Fahrenheit less than the melting point of the first biphasic film and the second biphasic film.

3. The clear soft vegetarian dosage capsule of claim 1, wherein the polysaccharide is a member of the group consisting of: alginates, agar gum, gellan, pullulan, starch, tara gum, locust bean gum, glucomannan, or monosaccharide gums and combinations thereof.

4. The clear soft vegetarian dosage capsule of claim 1, wherein the seaweed extract is a member of the group consisting of: carrageenan, red seaweed, brown seaweed, funoran algae, red algae, brown algae, green algae, kelp, other marine plants, and combinations thereof.

5. The clear soft vegetarian dosage capsule of claim 1, wherein the rheology modifier is a xanthan gum, arabic gum, and karaya gum, or combinations thereof.

6. The clear soft vegetarian dosage capsule of claim 1, wherein the buffer is a member of the group consisting of: potassium acetate, magnesium acetate, ethylene diaminetetraacetic acid, and combinations thereof.

7. The clear soft vegetarian dosage capsule of claim 1, wherein the plasticizer is a member of the group consisting of: glycerin, sorbitol, glycol and combinations thereof.

8. The clear soft vegetarian dosage capsule of claim 1, wherein the polysaccharide has a weight percent from 20 to 42, the seaweed exact has a weight percent from 5 to 24, the rheology modifier has weight percent from 7 to 22, the buffer has a weight percent from 0.01 to 10, and the plasticizer has weight percent from 10 to 40.

9. The clear soft vegetarian dosage capsule of claim 1, wherein each of the films further comprises from 0.01 to 60 weight percent of water.

10. The clear soft vegetarian dosage capsule of claim 1, wherein the capsule is a suppository, a wafer, a sublingual dissolvable film, a quick-dissolve ampule, a lozenge, or a strip.

11. The clear soft vegetarian dosage capsule of claim 1, further comprising a drug carrier.

12. The clear soft vegetarian dosage capsule of claim 11, wherein the drug carrier is a hard capsule.

13. The clear soft vegetarian dosage capsule of claim 11, wherein the drug carrier is a soft capsule.

14. The clear soft vegetarian dosage capsule of claim 1, wherein the dosage amount is a liquid.

15. The clear soft vegetarian dosage capsule of claim 14, wherein the liquid is a marine oil, a vitamin oil, or vegetable oil, synthetic oil, or combinations.

16. The clear soft vegetarian dosage capsule of claim 1, wherein the dosage amount is a pharmaceutical dosage amount.

17. The clear soft vegetarian dosage capsule of claim 16, wherein the pharmaceutical dosage amount is a liquid dosage amount that is a member of the group consisting of: aspirin, antihistamine, liquid delivery acetaminophen, liquid anti-inflammatory drugs, marine lipids, antibiotics, and combinations thereof.

18. The clear soft vegetarian dosage capsule of claim 1, wherein the dosage amount is a nutritional dosage amount.

19. The clear soft vegetarian dosage capsule of claim 18, wherein the nutritional dosage amount is a member of the group consisting of: Vitamin E, Vitamin B1, algae DHA, Vitamin A, Vitamin D, Vitamin B12, other Vitamin B mixtures, Vitamin B-6, Vitamin B3, flax seed oil, lecithin, omega oils, fish oils, evening primrose oil, borage oil, pumpkin seed oils, linoleic acis, lipds, silymarin, and combinations thereof.

20. The clear soft vegetarian dosage capsule of claim 1, wherein the dosage amount is a cosmetic.

21. The clear soft vegetarian dosage capsule of claim 20, wherein the cosmetic is a liquid foundation, a moisturizer, an antiaging product, a skin de-wrinkle component, or other minerals.

22. The clear soft vegetarian dosage capsule of claim 1, wherein at least one of the films further comprises a flavoring additive.

23. The clear soft vegetarian dosage capsule of claim 22, wherein the flavoring additive is a member of the group consisting of: citrus flavoring, mint flavoring, rum flavors, other fruit flavors, bubblegum flavoring, vanilla flavoring, cocoa flavoring, licorice flavoring, coffee flavoring, and combinations thereof

24. The clear soft vegetarian dosage capsule of claim 1, wherein at least one of the films further comprises an edible coloring.

25. The clear soft vegetarian dosage capsule of claim 24, wherein the edible coloring is a member of the group consisting of: azoquinophthalone triphenylmethane, xanthene, or indigoid dyes, iron oxides, iron hydroxides, titanium dioxides, carbon black, riboflavin, carotenes, anthocyanines, tumeric, cochineal extracts, chlorophyllin, canthaxanthin, caramel, betannin, and combinations thereof.

26. The clear soft vegetarian dosage capsule of claim 1, wherein each reservoir is a size from 100 mg to 1500 mg.

27. The clear soft vegetarian dosage capsule of claim 1, wherein each film has a thickness when dry, and after cooling from 0.05 cm to 0.09 cm.

28. The clear soft vegetarian dosage capsule of claim 1, wherein the plurality of clear soft vegetarian dosage capsules are formed simultaneously with cutting and sealing using a pressure from 0.25 psi to 1 psi.

29. The clear soft vegetarian dosage capsule of claim 1, wherein each film has an elasticity sufficient to allow each film to stretch during filling for use during continuous commercial processes.

30. The clear soft vegetarian dosage capsule of claim 1, wherein a shape of the reservoir is round, elliptical, or oblong.

31. The clear soft vegetarian dosage capsule of claim 1, further comprising a polysaccharide coating disposed over the clear soft vegetarian dosage capsules.

32. The clear soft vegetarian dosage capsule of claim 31, wherein the polysaccharide coating is different from the polysaccharide.
